(19) United States
(12) Patent Application Publication
(10) Pub. No.: US 2011/0070229 A1
(43) Pub. Date: Mar. 24, 2011

(54) Method of selecting a monoclonal antibody for administration

(75) Inventor: John Simard, Austin, TX (US)

(73) Assignee: XBIOTECH, INC., Vancouver (CA)

(21) Appl. No.: 12/888,534

(22) Filed: Sep. 23, 2010

Related U.S. Application Data
(60) Provisional application No. 61/245,305, filed on Sep. 24, 2009.

(51) Int. Cl. A61K 39/395; A61P 37/00

(52) U.S. Cl. 424/133.1; 424/142.1

(57) ABSTRACT

Methods, compositions, and kits relating to selecting a prophylactic or therapeutic antibody less likely to induce or aggravate an anti-antibody response in a subject administered the antibody. An antibody for administration to a subject may be selected to match, or at least more closely resemble, the allotypic phenotype of the subject's endogenous antibodies.

### CROSS-REFERENCE TO RELATED APPLICATIONS

The present application claims priority from U.S. provisional patent application No. 61/245,305 filed on Sep. 24, 2009 which is incorporated in its entirety herein by reference.

### FIELD OF THE INVENTION

The invention relates generally to the fields of immunology, antibodies (Abs), and medicine. More particularly, the invention relates to the selection of prophylactic or therapeutic Abs based on allotypic phenotype for the reduction of adverse reactions associated with anti-Ab responses.

### BACKGROUND

Therapeutic monoclonal Abs (mAbs) are the fastest growing segment of the pharmaceutical industry. So far, over 20 mAbs have been approved by the FDA for use as drugs, with many more in development. Although methods now exist for creating fully human mAbs, almost all of the FDA-approved therapeutic mAbs have been derived from rodents. Unfortunately, administration of non-human mAbs can cause serious, sometimes fatal, reactions.

Hypersensitivity reactions caused by administration of non-human Abs to human subjects have been known for several decades. Much like a vaccine against a disease causing microorganism, administration of non-human antibodies immunizes a person against these foreign glycoproteins. While a first administration of a non-human Ab is not usually associated with a vigorous anti-Ab response, repeated administration of non-human Abs for the treatment of a chronic condition can lead to serious side effects (including death) caused by the ensuing anti-Ab response.

This problem was particularly severe in early Ab-based treatments that used horse antiserum or murine mAbs. More recently, attempts have been made to reduce anti-Ab responses by modifying non-human Abs to make them appear more human. For example, a common practice for “humanizing” murine Abs involves replacing murine sequences outside a mAb's complementarity determining region (CDR) with actual human sequences. Although such CDR-grafting techniques have helped reduce human-anti-mouse Ab (HAMA) responses, because they do not eliminate all mouse sequences, they have not eliminated the problem.

A number of approaches have been used to develop so-called “human” Abs. These approaches include the use of mice genetically engineered to produce Abs from human gene sequences, as well the use of in vitro combinatorial approaches using DNA libraries. It is evident from clinical studies that therapeutic Abs derived from sequences that are more similar to those in human Abs result in delayed time of onset or intensity of anti-Ab-related side effects. The use of fully human Abs will almost certainly become standard practice in the future. Despite this, because of the large amount of genetic variation in the human population, human-anti-human Ab (HAHA) responses will prove difficult to eliminate.

### SUMMARY

The invention relates to the development of methods and compositions that reduce the likelihood that a subject will develop an anti-Ab response to an administered Ab. In humans, there are five different classes of Abs (or immunoglobulins; Igs), known as IgA, IgD, IgE, IgG and IgM. Different Abs within a single class have different variable regions but similarly structured constant regions. Within a class of Igs, there can be subclasses. For example, a human Ig of the IgG class can be of one of the four IgG subclasses: IgG₁, IgG₂, IgG₃, or IgG₄. The constant regions of any given subclass have almost identical amino acid sequences, while those of different subclasses have less similar amino acid sequences. While all normal human beings possess all Ig classes and subclasses, an Ig of the same class and subclass might exist in two or more allelic forms (allotypes) with some of the allelic forms being found in some people but not others. Within the IgG₁ subclass, for instance, there are four heavy chain alleles (or allotypes): G1m1, G1m2, G1m3, and G1m17; and three light (kappa) chain alleles: Km1; Km1,2; and Km3. The different IgG₁ alleles are defined according to small amino acid sequence variations in the constant regions. For example, otherwise identical in sequence, the difference between the constant regions of the G1m3 and G1m2 allotypes totals four amino acids.

An important drawback of conventional therapeutic Ab methods is that they do not take into account the allotypic phenotype of the subject's endogenous Abs. Thus, if a given mAb has allotypic determinants not expressed in a particular subject, the immune system of the subject will likely generate an anti-allotypic Ab (AAAb) response against the therapeutic mAb—a response that can lead to side effects such as a hypersensitivity reaction, or neutralization of the therapeutic effects of the mAb. An AAAb response might occur even where the subject was not previously administered the therapeutic mAb, e.g., where the subject has (i) naturally occurring AAAbs, (ii) a condition associated with high titers of anti-Abs (e.g., rheumatoid arthritis), (iii) AAAbs generated in response to a blood transfusion, (iv) maternally derived AAAbs, (v) AAAbs generated in response to a pregnancy, and/or (vi) AAAbs generated in response to another therapeutic mAb.

The invention relates to selecting an Ab to be administered to a subject to match, or at least more closely resemble, the allotypic phenotype of the subject's endogenous Abs to reduce Ab-induced side effects and neutralization. Because the immune system is geared to not produce a response to self antigens, administration of Abs that look to the immune system more like self Abs, are less likely to cause an anti-Ab response. Thus, the invention may make it possible to use a particular Ab for a longer time period than would otherwise be possible and/or without concomitant use of immunosuppressive drugs (e.g., methotrexate or steroids).

Accordingly, the invention features a panel of monoclonal antibody-containing pharmaceutical compositions. The panel including at least a first pharmaceutical composition including (a) a first human or humanized monoclonal antibody and a pharmaceutically acceptable carrier and (b) a second human or humanized monoclonal antibody and a pharmaceutically acceptable carrier, wherein the first monoclonal antibody is of a first isotype and includes a first variable region, and the second monoclonal antibody is of the first isotype and includes the first variable region, and wherein the first monoclonal antibody includes a first heavy chain allotypic phenotype and the second monoclonal antibody includes a second heavy chain allotypic phenotype differing from the first heavy chain allotypic phenotype.

The panel might further include (c) a third human or humanized monoclonal antibody and a pharmaceutically acceptable carrier, wherein the third monoclonal antibody is of the first isotype, includes the first variable region, and includes a third heavy chain allotypic phenotype differing from the first and second heavy chain allotypic phenotypes; and, in some cases, (d) a fourth human or humanized monoclonal antibody and a pharmaceutically acceptable carrier, wherein the fourth monoclonal antibody is of the first isotype, includes the first variable region, and includes a fourth heavy chain allotypic phenotype differing from the first, second, and third heavy chain allotypic phenotypes.

In the panel, the first isotype can be gamma 1, and, e.g., the first heavy chain allotypic phenotype might include the human G1m3 allotype and the second heavy chain allotypic phenotype might include the human G1m17 allotype. The first isotype might also be gamma 3, and, e.g., the first heavy chain allotypic phenotype might include the human G3m5 allotype and the second heavy chain allotypic phenotype might include the human G3m21 allotype. The first heavy chain allotypic phenotype and the second heavy chain might also include different allotypic phenotypes selected from the group consisting of: G3m5,10,11,13,14,26,27; G3m21,26,27,28; G3m10,11,13,15,27; G3m10,11,13,15,16,27; G3m5,6,10,11,14,26,27; and G3m5,6,11,24,26

In another aspect, the invention features a method of modifying a human or humanized monoclonal antibody. This method can include the step of modifying the amino acid sequence of the constant region of the heavy chain of the monoclonal antibody to change its phenotype from a first naturally occurring allotypic phenotype to a second naturally occurring allotypic phenotype, wherein the amino acid sequences in the monoclonal antibody that do not encode an allotypic phenotype are not modified. In this method, the monoclonal antibody can be an IgG₁ that is modified from a G1m1 allotype to a nG1m1 allotype; and/or modified from a G1m3 allotype to a G1m17 allotype. In the method, the second naturally occurring allotypic phenotype can be an isoallotype or non-marker. For example, where the monoclonal antibody is an IgG₂, it can be modified from a G2m23 allotype to an nG2m23 isoallotype.

Also within the invention is a method of selecting a human or humanized monoclonal antibody for administration to a human subject. This method can include the steps of: (a) determining the presence of a first antibody allotypic phenotype in the subject; and (b) selecting a monoclonal antibody to be administered to the subject from a set of human or humanized monoclonal antibodies including at least a first monoclonal antibody including the first allotypic phenotype and a second monoclonal antibody including a second allotypic phenotype not endogenously present in the subject; and (c) administering the first monoclonal antibody to the subject. In this method, the first monoclonal antibody can be one that does not include an allotypic phenotype not endogenously present in the subject. The first monoclonal antibody can be of a first isotype and include a first variable region, and the second monoclonal antibody can be of the first isotype and include the first variable region. The subject in this method can be one that has rheumatoid arthritis; has been previously transfused with heterologous blood; is a female who has been pregnant; is a child less than one year of age; and/or has been previously administered an antibody including the second allotypic phenotype.

The invention further features a method of selecting a monoclonal antibody for administration to a human subject belonging to a defined population (e.g., the White, Black, or Asian population). This method can include the steps of: (a) determining a defined population that includes the subject; (b) selecting a monoclonal antibody for administration to the subject from a set of human or humanized monoclonal antibodies including at least a first monoclonal antibody including a first allotypic phenotype more common in the defined population and a second monoclonal antibody including a second allotypic phenotype less common in the defined population than the first allotypic phenotype; and (c) administering the first monoclonal antibody to the subject. In this method, the first monoclonal antibody can include a first haplotype more common in the defined population and the second monoclonal antibody can include a second haplotype less common in the defined population than the first haplotype. The defined population is the White population. In this method, the subject can be one that has rheumatoid arthritis; has been previously transfused with heterologous blood; is a female who has been pregnant; is a child less than one year of age; and/or has been previously administered an antibody including the second allotypic phenotype.

In another aspect, the invention features a method of treating a subject who has developed an anti-antibody response to a first human or humanized monoclonal antibody. This method can include the step of administering to the subject a second human or humanized monoclonal antibody having a variable region identical to the first monoclonal antibody but a different allotypic phenotype than the first monoclonal antibody. The anti-antibody response can be one characterized by the presence of antibodies in the subject that specifically bind to allotypic determinants of the first monoclonal antibody. The second monoclonal antibody can be one that does not include an allotypic phenotype not endogenously present in the subject

Also within the invention is a method of screening a subject for the presence of antibodies that specifically bind an allotypic determinant not endogenously expressed by the subject. This method can include the steps of: (a) obtaining a biological sample from the subject; (b) contacting the biological sample with a probe specific for the anti-allotypic determinant; and (c) detecting binding of the probe to antibodies that might be contained in the biological sample as an indication that the subject harbors antibodies that specifically bind the allotypic determinant not endogenously expressed by the subject. The probe can be an antibody that includes the allotypic determinant not endogenously expressed by the subject.

Further within the invention is a kit for characterizing the allotypic phenotype of a subject. The kit can include: at least a first probe that specifically identifies a first antibody allotypic determinant and a second probe that specifically identifies a second antibody allotypic determinant differing from the first antibody allotypic determinant; at least a first positive control including a first molecule including or encoding the first allotypic determinant and a second positive control including a second molecule including or encoding the second allotypic determinant; at least a first negative control including a third molecule not including or encoding the first allotypic determinant and a second positive control including a fourth molecule not including or encoding the second allotypic determinant; and printed instructions for using the kit. The first and second probes can be antibodies that specifically bind a different allotypic determinant selected from the group consisting of: G1m1, G1m2, G1m3, G1m17, G3m5, G3m6, G3m10, G3m11, G3m13, G3m14, G3m15, G3m16, G3m21, G3m24, G3m26, G3m27, and G3m28. The kit can further include a plurality of polymerase chain reaction primers that specifically amplify different allotypic determinants, the different allotypic determinants being selected from the group consisting of G1m1, G1m2, G1m3, G1m17, G3m5, G3m6, G3m10, G3m11, G3m13, G3m14, G3m15, G3m16, G3m21, G3m24, G3m26, G3m27, and G3m28.

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994.

As used herein, an “antibody” or “Ab” is an Ig, a solution of identical or heterogeneous Igs, or a mixture of Igs. Engineered Igs such as diabodies and immunoadhesins that contain at least one Ig allotypic determinant are also considered “Abs.” A “monoclonal antibody” or “mAb” is an Ab expressed by one clonal B cell line. As used herein, the term refers to a population of Ab molecules that contains only one species of an antigen binding site capable of immunoreacting with a particular epitope of a particular antigen. A “polyclonal antibody” or “polyclonal Ab” is a mixture of heterogeneous Abs. Typically, a polyclonal Ab will include myriad different Ab molecules which bind a particular antigen or particular organism with at least some of the different Abs immunoreacting with a different epitope of the antigen or organism. As used herein, a polyclonal Ab can be a mixture of two or more mAbs.

By the phrase “allotypic determinant” is meant an endogenous amino acid sequence located on a location of an Ig corresponding to the site defining an allotype.

As used herein, the phrase “allotypic phenotype” means an amino acid sequence(s) of an Ab defining an allotype or the amino acid sequence(s) at the same position as that defining an Ab allotype. The phrase allotypic phenotype can include an antibody with a single allotype, isoallotype, or non-marker; or more than one (e.g., 2, 3, 4, 5, 6, or more) allotype, isoallotype, or non-marker.

An “antigen-binding portion” of an Ab is contained within the variable region of the Fab portion of an Ab and is the portion of the Ab that confers antigen specificity to the Ab (i.e., typically the three-dimensional pocket formed by the CDRs of the heavy and light chains of the Ab). A “Fab portion” or “Fab region” is the proteolytic fragment of a papain-digested Ig that contains the antigen-binding portion of that Ig. A “F(ab′)₂ portion” is the proteolytic fragment of a pepsin-digested Ig. A “Fab′ portion” is the product resulting from reducing the disulfide bridges of an F(ab′)₂ portion. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is generally an effector portion of that Ab.

When referring to a protein molecule such as an Ab, “purified” means separated from components that naturally accompany such molecules. Typically, an Ab or protein is purified when it is at least about 10% (e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is naturally associated. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.” An Ab containing a desired Ig type and an undesired Ig type is “enriched” for the desired Ig type when treatment of the Ab results in a higher ratio of desired Ig to undesired Ig after treatment than before treatment. For example, a solution of Ab containing IgGs of allotype G1m3 and G1m17 is enriched for the latter when some or all of the IgG of the allotype G1m3 are removed from the solution.

By “bind”, “binds”, or “reacts with” is meant that one molecule recognizes and adheres to a particular second molecule in a sample, but does not substantially recognize or adhere to other molecules in the sample. Generally, an Ab that “specifically binds” another molecule has a K_{d} greater than about 10⁵, 10⁶, 10⁷, 10⁸, 10⁹, 10¹⁰, 10¹¹; or 10¹² liters/mole for that other molecule.

As used herein, the term “polypeptide” encompasses native or artificial proteins, protein fragments and polypeptide analogs of a protein sequence. A polypeptide may be monomeric or polymeric.

A “human Ab” is an Ab having variable and constant regions derived from human germline Ig sequences. Human antibodies may include amino acid residues not encoded by human germline Ig sequences (e.g., mutations introduced by random or site-specific mutagenesis in vitro or by somatic mutation in vivo), for example in the CDRs and in particular CDR3. A “human Ab”, however, does include an Ab with CDR sequences derived from the germline of a non-human species and grafted onto human framework sequences (which is a “humanized Ab”).

The term “recombinant Ab” is intended to include all Abs that are prepared, expressed, created or isolated by recombinant means, such as Abs expressed using a recombinant expression vector transfected into a host cell, Abs isolated from a recombinant, combinatorial Ab library, Abs isolated from an animal (e.g., a mouse) that is transgenic for human Ig genes (see e.g., Taylor, L. D., et al. (1992) Nucl. Acids Res. 20:6287-6295) or Abs prepared, expressed, created or isolated by any other means that involves splicing of Ig gene sequences to other DNA sequences.

The term “epitope” includes any antigenic determinant capable of specific binding to an Ig.

A “therapeutically effective amount” is an amount which is capable of producing a medically desirable effect in a treated animal or human (e.g., amelioration or prevention of a disease).

As used herein, the term “subject” means any Ab-containing animal including mammals such as human beings, dogs, cows, horses, pigs, sheep, goats, cats, mice, rabbits, rats, and transgenic non-human animals.

The term “pharmaceutical composition” or “medicament” refers to preparations which are in such form as to permit the biological activity of the active ingredients to be effective. “Pharmaceutically acceptable excipients” or “pharmaceutically acceptable carriers” are substances, which can be mixed with one or more active ingredients to form a medication which can reasonably (i.e., safely) be administered to a subject to provide an effective dose of the active ingredient(s) employed.

When referring to defined populations, the term “White” means a person having origins in any of the original peoples of Europe, the Middle East, or North Africa; the term “Black” refers to a person having origins in any of the Black racial groups of Africa; the terms “American Indian and Alaska Native” mean a person having origins in any of the original peoples of North and South America (including Central America); and the term “Asian” means a person having origins in any of the original peoples of the Far East, Southeast Asia, or the Indian subcontinent.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the materials, methods, and examples are illustrative only and not intended to be limiting.

Other features and advantages of the invention will be apparent from the following detailed description, and from the claims.

### DETAILED DESCRIPTION

The invention encompasses methods, compositions, and kits relating to selecting a prophylactic or therapeutic Ab less likely to induce or aggravate an anti-Ab response in a subject administered the Ab. An Ab for administration to a subject may be selected to match, or at least more closely resemble, the allotypic phenotype of the subject's endogenous Abs. The below described preferred embodiments illustrate adaptation of these compositions and methods. Nonetheless, from the description of these embodiments, other aspects of the invention can be made and/or practiced based on the description provided below.

### Biological Methods

Methods involving conventional immunological and molecular biological techniques are described herein. Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Ab methods are described in Handbook of Therapeutic Abs, Dubel, S., ed., Wiley-VCH, 2007. Cell culture techniques are generally known in the art and are described in detail in methodology treatises such as Culture of Animal Cells: A Manual of Basic Technique, 4th edition, by R Ian Freshney, Wiley-Liss, Hoboken, N.J., 2000; and General Techniques of Cell Culture, by Maureen A Harrison and Ian F Rae, Cambridge University Press, Cambridge, UK, 1994. Methods of protein purification are discussed in Guide to Protein Purification: Methods in Enzymology, Vol. 182, Deutscher M P, ed., Academic Press, San Diego, Calif., 1990.

### Subjects

The methods, compositions, and kits of the invention are for use with animal subjects including human beings and other mammals such as cats, dogs, mice, rats, rabbits, sheep, cows, horses, goats, pigs, monkeys, and apes. The invention is particularly useful for those subjects having measureable titers (e.g., greater than 0.001, 0.01, 0.05, 0.1, 0.2, 0.5, or 1 μg Ab per ml of serum) of AAAb or those at high risk for developing AAAbs, e.g., subjects having HAMA, HAHA, or a condition associated with high titers of anti-Abs (e.g., an autoimmune disease such as rheumatoid arthritis); subjects who are or have been pregnant; children who have maternal Abs (e.g., those less than one year of age); subjects who have had a blood transfusion; subjects having naturally occurring AAAbs; and subjects having AAAbs generated in response to another therapeutic Ab.

### Antibodies

The methods, compositions, and kits described herein can use or include various different types of Abs including mAbs, polyclonal Abs, and various Abs fragments (e.g., Fab fragments, Fab′ fragments, and F(ab′)₂ fragments), or engineered Abs (e.g., single chain antibodies, and molecules produced using a Fab expression library) that (i) retain an endogenous amino acid sequence at the location of at least one (e.g., 2, 3, 4, 5, or more) allotypic determinant, (ii) include amino acid sequences from heavy and/or light chain Igs that do not have allotypic variants, and/or (iii) include amino acid sequences from heavy and/or light chain Igs that define an isoallotype or non-marker.

MAbs, which are homogeneous populations of antibodies to a particular antigen, can be prepared using standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, N.Y., 1981; Ausubel et al., supra). In particular, MAbs can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). MAbs may also be isolated from phage antibody libraries using the techniques described in Clackson, et al., Nature 352:624-628 (1991) and Marks, et al., J. Mol. Biol. 222:581-597 (1991). A cell line producing a mAb may be cultivated in vitro or in vivo to produce large amounts of mAbs.

Polyclonal Abs are heterogeneous populations of Ab molecules that are contained in the sera of the immunized subjects or combinations of different mAbs. Polyclonal Abs can be isolated by collecting sera from immunized host animals according to conventional methods. Polyclonal antibodies collected from serum are generally heterogeneous with respect to allotype, but can be used to prepare polyclonal Abs having identical allotypic determinants, e.g., by immunoaffinity purification using immobilized Abs specific for the allotypic determinant(s).

Single-chain Abs against a target antigen can be prepared by conventional methods (e.g., U.S. Pat. Nos. 4,946,778, 4,946,778, and 4,704,692), e.g., by linking heavy and light chain fragments of an Fv region via an amino acid bridge, resulting in a single chain polypeptide (scFv). Ab fragments that specifically bind a target antigen can also be prepared by conventional techniques. For example, Fab fragments can be produced by papain digestion of a full-length Ig, F(ab′)₂ fragments can be produced by pepsin digestion of a full-length Ig molecule, and Fab′ fragments can be generated by reducing the disulfide bridges of F(ab′)₂ fragments. Fab expression libraries can be constructed and screened by known methods (e.g., Huse et al., Science 246:1275, 1989) to produce monoclonal Fab fragments with a desired specificity. Diabodies (i.e., bivalent Abs in which V_{H} and V_{L} domains are on a single polypeptide chain) can be produced by known methods (e.g., Holliger P. et al., Proc. Natl. Acad. Sci. USA 90:6444-6448 (1993) and Poljak R. J. et al., Structure 2:1121-1123 (1994)). Immunoadhesins which contain portions of Abs (e.g., portions of Ig heavy chains) fused to a non-Ab molecule (e.g., a cytokine or cytokine receptor) might also be used in the invention.

Abs can be purified by conventional techniques including: salt cuts (e.g., saturated ammonium sulfate precipitation), cold alcohol fractionation (e.g., the Cohn-Oncley cold alcohol fractionation process), size exclusion chromatography, ion exchange chromatography, immunoaffinity chromatography (e.g., chromatography beads coupled to anti-human Ig antibodies can be used to isolate human Igs), Protein A or Protein G chromatography, and antigen affinity chromatography. See, e.g., Coligan et al., supra.

Standard techniques in immunology and protein chemistry can be used to analyze and manipulate Abs. For example, dialysis can be used to alter the medium in which Abs are dissolved. Ab may also be lyophilized for preservation. Abs can be tested for the ability to bind specific antigens using any one of several standard methods such as Western blot, immunoprecipitation analysis, enzyme-linked immunosorbent assay (ELISA), and radioimmunoassay (RIA). See, e.g., Coligan et al., supra.

The various Abs described herein might be conjugated to another molecule such as a detectable label, a cytotoxic agent or a radioisotope. Examples of the detectable labels may include chromogenic enzymes (such as peroxidase and alkaline phosphatase), radioisotopes (such as ¹²⁴I, ¹²⁵I, ¹¹¹In, ⁹⁹mTc, ³²P, and ³⁵S), chromophores, biotins, and luminescent or fluorescent dyes (such as FITC, RITC, rhodamine, Texas Red, fluorescein, phycoerythrin, dye-doped nanoparticles, and quantum dots), MR contrast agents (such as superparamagnetic iron oxides (SPIO) and ultrasuperparamagnetic iron oxides (USPIO)). Examples of cytotoxic agents include, without limitation, radioisotopes (e.g., ³⁵S, ¹⁴C, ³²P, ¹²⁵I, ¹³¹I, ⁹⁰Y, ⁸⁹Zr, ²⁰¹Tl, ¹⁸⁶Re, ¹⁸⁸Re, ⁵⁷Cu, ²¹³Bi, and ²¹¹At), conjugated radioisotopes, antimetabolites [e.g., 5-fluorouricil (5-FU), methotrexate (MTX), fludarabine, etc.], anti-microtubule agents [e.g., vincristine, vinblastine, colchicine, and taxanes (such as paclitaxel and docetaxel)], alkylating agents [e.g., cyclophasphamide, melphalan, and bischloroethylnitrosurea (BCNU)], platinum agents [e.g., cisplatin (also termed cDDP), carboplatin, and oxaliplatin], anthracyclines (e.g., doxorubicin and daunorubicin), antibiotic agents (e.g., mitomycin-C), topoisomerase inhibitors (e.g., etoposide, tenoposide, and camptothecins), or other cytotoxic agents such as ricin, diptheria toxin (DT), Pseudomonas exotoxin (PE) A, PE40, abrin, saporin, pokeweed viral protein, ethidium bromide, glucocorticoid, anthrax toxin and others. See, e.g., U.S. Pat. No. 5,932,188.

### Target Antigens

The Abs described herein are generally selected to specifically bind to a target antigen, although non-antigen specific Abs such as intravenous immunoglobulin compositions prepared to match, or at least more closely resemble, the allotypic phenotype of a subject's endogenous Abs are also within the invention. Examples of target antigens include molecules expressed on a cell surface; molecules expressed within a cell; molecules present in a bodily fluid or tissue; molecules expressed by a bacteria, virus, or parasite; drugs; and poisons. These include, without limitation, adhesion molecules, CD antigens, receptors, cytokines, cytokine receptors, enzymes, enzyme co-factors, or DNA-binding proteins such as ApoE, Apo-SAA, BDNF, Beta amyloid, CA125, cardiac myosin, Cardiotrophin-1, cancer-associated antigens, CD1 (a-c, 1A, 1D, 1E), CD2, CD3 (y, 6, E), CD4, CD5, CD6, CD7, CD8, CD9, CD10, CD11 (a, b, c), CD13, CD14, CD15, CD16 (A, B), CD18, CD19, CD20, CD21, CD22, CD23, CD24, CD25, CD26, CD27, CD28, CD29, CD30, CD31, CD32 (A, B), CD33, CD34, CD35, CD36, CD37, CD38, CD39, CD40, CD41, CD42 (a, b, c, d), CD43, CD44, CD45, CD46, CD47, CD48, CD49 (a, b, c, d, e, f), CD50, CD51, CD52, CD53, CD54, CD55, CD56, CD57, CD58, CD59, CD61, CD62 (E, L, P), CD63, CD64 (A, B, C), CD66 (a, b, c, d, e, f), CD68, CD69, CD70, CD71, CD72, CD73, CD74, CD78, CD79 (a, b), CD80, CD81, CD82, CD83, CD84, CD85 (a, d, e, h, j, k), CD86, CD87, CD88, CD89, CD90, CD91, CD92, CD93, CD94, CD95, CD97, CD98, CD99, CD100, CD101, CD102, CD103, CD104, CD105, CD106, CD107 (a, b), CD108, CD109, CD110, CD111, CD112, CD113, CD114, CD115, CD116, CD117, CD118, CD119, CD120 (a, b), CD121 (a, b), CD122, CD123, CD124, CD125, CD126, CD127, CD129, CD130, CD131, CD132, CD133, CD134, CD135, CD136, CD137, CD138, CD140b, CD141, CD142, CD143, CD144, CD146, CD147, CD148, CD150, CD151, CD152, CD153, CD154, CD155, CD156 (a, b, c), CD157, CD158 (a, d, e, i, k), CD159 (a, c), CD160, CD161, CD162, CD163, CD164, CD166, CD167 (a, b), CD168, CD169, CD170, CD171, CD172 (a, b, g), CD174, CD177, CD178, CD179 (a, b), CD181, CD182, CD183, CD184, CD185, CD186, CD191, CD192, CD193, CD194, CD195, CD196, CD197, CDw198, CDw199, CD200, CD201, CD202b, CD204, CD205, CD206, CD207, CD208, CD209, CDw210 (a, b), CD212, CD213a (1, 2), CD217, CD218 (a, b), CD220, CD221, CD222, CD222, CD224, CD225, CD226, CD227, CD228, CD229, CD230, CD233, CD234, CD235 (a, b), CD236, CD238, CD239, CD240CE, CD241, CD243, CD244, CD246, CD247, CD248, CD249, CD252, CD253, CD254, CD256, CD257, CD258, CD261, CD262, CD264, CD265, CD266, CD267, CD268, CD269, CD27, CD272, CD273, CD274, CD275, CD276, CD278, CD279, CD280, CD281, CD282, CD283, CD284, CD286, CD288, CD289, CD290, CD292, CDw293, CD294, CD295, CD297, CD298, CD299, CD300A, CD301, CD302, CD303, CD304, CD305, CD306, CD30, CD309, CD312, CD314, CD315, CD316, CD317, CD318, CD320, CD321, CD322, CD324, CD325, CD326, CD328, CD329, CD331, CD332, CD333, CD334, CD335, CD336, CD337, CD338, CD339, CD340, CD344, CD349, CD350, CEACAM3, CGM1, a CMV antigen, complement (e.g., C5), CTLA4, digoxin, EGF, EGF receptor, ENA-78, endotoxin, Eotaxin, Eotaxin-2, Exodus-2, Factor VII, FGF-acidic, FGF-basic, fibrin, fibroblast growth factor-10, FLT3 ligand, FOLR1, Fractalkine (CX3C), GCP-2, GD2 ganglioside, GDNF, G-CSF, GM-CSF, GF-beta1, GRO/MGSA, GRO-beta, GRO-gamma, an HBV antigen, an HCV antigen, HCC1, 1-309, a heat shock protein, HER 1, HER 2, HER 3, HER 4, a herpes virus antigen, an HIV antigen, HLA, HMW-MAA, an HSV antigen, insulin, IFN-gamma, IgE, IGF-I, IGF-II, IGF-1R, IL-1alpha, IL-1beta, IL-2, IL-3, IL-4, IL-5, IL-6, IL-7, IL-8 (72 a.a.), IL-8 (77 a.a.), IL-9, IL-10, IL-11, IL-12, IL-13, IL-15, IL-16, IL-17, IL-18 (IGIF), Inhibin alpha, Inhibin beta, IP-10, IRP-2, keratinocyte growth factor-2 (KGF-2), KGF, Lewis Y, lipoteichoic acid, Leptin, LIF, Lymphotactin, Lysozyme, Mullerian inhibitory substance, monocyte colony inhibitory factor, monocyte attractant protein, M-CSF, MDC (67 a.a.), MDC (69 a.a.), MCP-1 (MCAF), MCP-2, MCP-3, MCP-4, MDC (67 a.a.), MDC (69 a.a.), MIG, MIP-1alpha, MIP-1beta, MIP-3alpha, MIP-3beta, MIP-4, MUC1, myeloid progenitor inhibitor factor-1 (MPIF-1), NAP-2, NCA 90, Neurturin, Nerve growth factor, beta-NGF, NT-3, NT-4, Oncostatin M, PDGF-AA, PDGF-AB, PDGF-BB, PF-4, Phosphatidylserine, PSA, PSCA, PSMA, a rabies virus antigen, RANTES, RSV, SDF1alpha, SDF1beta, SCF, SCGF, stem cell factor (SCF), TACSTD1, TAG 72, TARC, TACE recognition site, tenacsin C, TGF-alpha, TGF-beta, TGF-beta2, TGF-beta3, tumor necrosis factor (TNF), TNF-alpha, TNF-beta, TNF receptor I (p55), TNF receptor II, TNIL-1, TPO, TRAIL-R1, VEGF, VEGF-A, VEGF receptor 1, VEGF receptor 2, VEGF receptor 3, and receptors of the foregoing. Muromonab-CD3, edrecolomab, ibritumomab tiuxetanm, tositumomab, abciximab, rituximab, basiliximab, infliximab, cetuximab, daclizumab, palivizumab, trastuzumab, gemtuzumab, alemtuzumab, omalizumab, efalizumab, bevacizumab, nimotuzumab, natalizumab, ranibizumab, eculizumab, certolizumab pegol, adalimumab, panitumumab, etanercept, alefacept, abatacept, and rilonacept also define epitopes for Abs useful in the invention.

### Ig Allotypes

The methods and compositions of the invention relate to specifically matching, or at least partially matching (e.g., at least more than 50, 60, 70, 80, 90, 95% matching) the allotypic phenotype of the therapeutic mAb to that of the subject. Various allotypes are known in the art. Table 1 (below) lists different human Ig alleles.

TABLE 1

| Ig Family | Alleles |
|---|---|
| Kappa light chain | Km1; Km1,2; Km3 |
| Lambda light chain | None |
| G1 | G1m1, nGm1 |
| | G1m2, nGm2 |
| | Gm3, Gm17 |
| G2 | G2m23, nG2m23 |
| G3 | G3m21, nG3m21 |
| | G3m5, nGm5 |
| | G3m11, nGm11 |
| | G3m6, G3m10, G3M13, |
| | G3m14, G3m15, G3m16, |
| | G3m21, G3m24, G3m26, |
| | G3m27, G3m28 |
| G4 | none |
| A1 | none |
| A2 | A2m1, A2m2 |
| D | none |
| M | none |
| E | none |

### Panel of Abs

The compositions, methods and kits of the invention can feature or utilize a panel of Abs including at least (i) a first Ab including a first heavy chain variable region and a first heavy chain constant region of a first allotypic phenotype and (ii) a second Ab including a heavy chain variable region identical to (or at least with the same antigen-binding specificity, e.g., in the case of a polyclonal Ab) that of the first Ab and a second heavy constant region of a second allotypic phenotype differing from the first allotypic phenotype. In preferred embodiments the panel of Abs includes several different human or humanized mAbs (e.g., 3, 4, 5, 6, 7, 8, 9, 10 or more different mAbs) each having identical variable regions (or antigen binding portions) but different constant regions, the constant regions being of the same isotype but differing in allotypic phenotype. For example, a panel of human or humanized mAbs might include two or three Igs with the same variable regions (and optionally the same heavy chain constant regions) but different kappa light chain constant regions, the differences in the kappa light chain constant regions corresponding to the Km1; Km1,2; and/or Km3 allotypes.

As another example, a panel of human or humanized mAbs might include at least two (e.g., 2, 3, or 4) IgG₁s with identical light chains and heavy chain variable regions, but different heavy chain constant regions, the differences in the heavy chain constant regions corresponding to the G1m1, G1m2, G1m3, and/or G1m17 allotypes. Because sets of allotypes are often inherited together, a panel of human or humanized Abs might include series of IgG₁s with identical light chains and heavy chain variable regions, but different but common heavy chain constant regions haplotypes such as two or three of G1m3; Gm1,17; and Gm1,2,17. A preferred set of human or humanized IgG₁ mAbs would include identical lambda light chains and heavy chain variable regions and (a) a heavy chain constant region of the nG1m1, nG1m2, and G1m3 allotypes and (b) a heavy chain constant region of the nG1m1, nG1m2, and G1m17 allotypes because this would include only two different possible antigenic sites (i.e., G1m3 and G1m17).

Similarly, a panel of human or humanized IgG₃ mAbs might include at least two (e.g., 2, 3, 4, 5, 6, 7, 8, 9, 10, or more) IgG₃s with identical light chains and heavy chain variable regions, but different heavy chain constant regions, the differences in the heavy chain constant regions corresponding to one or more (e.g., 1, 2, 3, 4, 5, 6, 7, 8, 9, 10 or more) of the G3m21, nG3m21, G3m5, nGm5, G3m11, nGm11, G3m6, G3m10, G3m13, G3m14, G3m15, G3m16, G3m21, G3m24, G3m26, G3m27, and G3m28 phenotypes. Because sets of allotypes are often inherited together, a panel of human or humanized Abs might include series of IgG₃s with identical light chains and heavy chain variable regions, but different but common heavy chain constant regions haplotypes such as two or more (e.g., 2, 3, 4, 5, or 6) of G3 m5,10,11,13,14,26,27; G3m21,26,27,28; G3m10,11,13,15,27; G3m10,11,13,15,16,27; G3 m5,6,10,11,14,26,27; and G3 m5,6,11,24,26. A panel of mAbs might include two human or humanized IgA₂s with identical light chains and heavy chain variable regions, but different heavy chain constant regions, the differences in the heavy chain constant regions corresponding to the Am1 and Am2 allotypes.

The Abs in the foregoing heavy chain constant region panels could include kappa and/or lambda light chains. Because the human lambda light chain does not contain allelic variants, use of only this light chain is preferred to reduce panel complexity. For kappa chain containing panels, each heavy chain allotype could be combined with kappa chains of either the Km1; Km1,2; and/or Km3 allotypes. In the case where different kappa chain allotypes are not included in a panel, Km3 is the most preferred light chain to use because it is the most common and Km1 is the least preferred because it is the least common. The panel of Abs of the invention could be arranged as a series of Ab-containing containers or vials.

### Antibodies with Reduced Immunogenicity

Also within the invention are Abs with reduced immunogenicity. Prophylactic or therapeutic Abs that generally exhibit reduced immunogenicity throughout members of a species are selected on the basis of allotypic phenotype. Those Abs with no or fewer potentially antigenic determinants associated with allotypic determinants are preferred. For example, human or humanized Abs that use lambda light chains (as opposed to kappa light chains) are preferred because they do not have allotypic determinants. Among kappa chains, those with the highest frequency in the population are preferred (Km3>Km1,2>Km1) to avoid an AAAb response. For human heavy chains, use of alpha 1, mu, epsilon, gamma4 (which has not allotypes) or gamma 2 with the nG2m23 isoallotype is preferred as no AAAb response should be generated against these chains. Gamma1 and gamma3, which exhibit the most allotypic variation, are however preferred formany applications (e.g., where good complement activation or ADCC is desired). In this case, gamma1 with nG1m1, nG1m2, and G1m3; or nG1m1, nG1m2, and G1m17 is preferred. The amino acid sequences in such heavy chains might also be engineered to replace the residues that define G1m3 or G1m17 with amino acids that define an isoallotypic determinant. Such engineered heavy chains can be used in Abs, and the Abs tested for desired activity (e.g., complement activation and ADCC activity).

### Methods for Selecting a Monoclonal Antibody for Administration

In one aspect, the invention features a method for selecting an Ab from a panel of different Abs, each having the same variable regions (or at least having the same antigen-binding specificity), being of the same isotype, but differing from one another by allotypic phenotype. This method includes the steps of: (a) obtaining a biological sample from a subject; (b) analyzing the biological sample to determine one or more (e.g., 1, 2, 3, 4, 5, 6, or more) Ab allotypic phenotypes expressed by the subject; (c) providing a panel of Abs including at least (i) a first Ab having a first variable region having an antigen binding portion specific for a predetermined antigen and a first constant region of a first allotypic phenotype not expressed by the subject and (ii) a second Ab having a first variable region having an antigen binding portion specific for the predetermined antigen (which could be identical to the variable region of the first Ab) and a second constant region of a second allotypic phenotype differing from the first allotypic phenotype and expressed by the subject; and (d) administering the second Ab to human subject. In this method the second constant region could also have multiple (e.g., at least 2, 3, 4, 5, 6, 7, 8, 9, 10 or more) allotypic phenotypes expressed by the subject and/or no allotypes not expressed by the subject.

Any suitable method for obtaining a biological sample might be employed. Biological samples obtained include any that contain an Ab or a cell having a nucleic acid encoding an Ab from a subject, e.g., blood, plasma, serum, white blood cells, B lymphocytes, cerebrospinal fluid, synovial fluid, spleen, lymph node, bone marrow, and placenta. For example, a peripheral blood sample can be obtained by venipuncture. Whole blood might be separated into a cell fraction (e.g., buffy coat), plasma, or serum according to known techniques. Bone marrow can be obtained by needle aspiration. Spleen and lymph node samples can be obtained by biopsy. In some cases, biological samples obtained from a subject can be further processed to enrich for Ab-containing portions or cells having a nucleic acid sequences encoding Ab.

To match an allotypic phenotype of an Ab to a subject, one or more (e.g., at least 2, 3, 4, 5, 6, 7, 8, 9, 10 or more) allotypic phenotypes of at least one (e.g., 1, 2, 3, 4, 5 or more) endogenous Ab in the subject can be determined. An Ab's allotypic phenotype can be determined by any suitable method, e.g., by identifying the amino acid sequence variation of an Ab corresponding to an allotypic phenotype in a biological sample taken from the subject or by determining the nucleic acid sequence of an Ab-encoding gene corresponding to an allotypic phenotype of DNA or RNA contained in a biological sample taken from the subject. For example, a probe specific for an amino acid variation associated with an allotypic phenotype can be contacted to an Ab-containing sample, and binding of the probe to the Ab can be assessed, wherein binding of the probe to the Ab indicates the sample contains Ab of that specific allotypic phenotype. As another example, a probe specific for a nucleic acid encoding amino acid variation associated with an allotypic phenotype can be contacted to a nucleic acid sample taken from the subject, and binding of the probe to the nucleic acid encoding the amino acid variation associated with an allotypic phenotype can be detected (e.g., by Southern blotting), wherein binding of the probe to the nucleic acid encoding amino acid variation associated with an allotypic phenotype indicates the subject has an Ab of that specific allotypic phenotype.

In a typical method, a biological sample such as blood, serum, or plasma is isolated from a subject to be Ab phenotyped. The isolated sample may be further processed to enrich Abs in the sample, e.g., by salt cuts, size exclusion chromatography, ion exchange chromatography, immunoaffinity chromatography (e.g., chromatography beads coupled to anti-human Ig antibodies can be used to isolate human Igs), and/or Protein A or Protein G chromatography. Immunoassays such as an enzyme-linked immunosorbent assay (ELISA) or a radioimmunoassay (RIA) can be used to determine the presence of Abs of a particular allotypic phenotype in a sample containing Abs of unknown allotypic phenotype. Such immunoassays typically employ Abs to selectively bind only Abs of a particular allotypic phenotype and a detectable label. For example, a capture Ab specific for a selected allotypic phenotype can be immobilized in wells of a microtiter plate. An Ab-containing biological sample such as diluted serum is added to the wells and the wells are washed. An enzyme-labeled Ab specific for the isotype of Ab being assessed (e.g., peroxidase-labeled anti-human IgG₁) is then added to the wells. The wells are then washed, and a substrate for the enzyme is added such that a colored reaction can be detected in wells where the Ab-containing biological sample included Abs of the particular allotypic phenotype being assessed.

Alternatively, a hemagglutination inhibition assay might be used. In an exemplary hemagglutination assay, O+ red cells are coated with Igs specific for a single known allotype such that the coated cells agglutinate when exposed to Ab of the single known allotype. Antiserum known to contain Abs of the single known allotype is mixed with an unknown serum to be typed, and the mixture is added to the coated red cells. If red cell agglutination is inhibited, it can be concluded that the allotype was present in the unknown serum.

In other techniques, the presence in a biological sample of a nucleic acid encoding amino acid variation associated with an allotypic phenotype can be detected. For example, a polymerase chain reaction (PCR)-based assay may also be used to determine expression of an Ab allotypic phenotype by a subject using primer sets that selectively amplify those nucleic acid regions encode the amino acid sequences in an Ab specific to a particular allotypic phenotype. In addition, restriction fragment length polymorphism analysis might be used to determine the allotype(s) expressed by a given subject. Although currently more cumbersome than immunoassays or nucleic acid-based detection techniques, an Ab or portion thereof might also be sequenced directly to determine its allotypic phenotype.

### Methods of Modifying an Ab

The invention further includes a method of modifying an Ab to change its allotypic phenotype while preserving its antigen-binding variable region. This method can be used to make a panel of Abs as described above. Methods of modifying Abs are well known in the art. See, e.g., Lo, B. K. C., Antibody Engineering-Methods and Protocols, Humana Press, 2004. Modifying an Ab is generally done using conventional molecular biology techniques where, for example, nucleic acids encoding whole Ig light or heavy chains are first isolated and cloned into a vector. Restriction enzymes are then used to cut out the nucleic acid sequences encoding the amino acid sequences corresponding to one or more (e.g., 1, 2, 3, 4, 5 or more) of the allotypic phenotypes of the Ab. The deleted nucleic acids are then replaced with new nucleic acid sequences encoding the amino acid sequence of one or more (e.g., 1, 2, 3, 4, 5 or more) different allotypic phenotypes. This process can be repeated to generate a library of nucleic acids that encode Abs with an identical variable region but different constant regions corresponding to different allotypic phenotypes of the Ab. Alternatively, CDR grafting wherein CDRs from an Ab with known antigen specificity is grafted onto framework regions of Abs of different allotypic phenotypes might be used. Using these methods, for example, an Ab of allotype G1m(1) can be changed to a G1m(3) allotype.

### Methods of Selecting a Monoclonal Antibody to a Human Subject Belonging to a Defined Population

The invention further features a method of selecting a monoclonal antibody for administration to a human subject belonging to a defined population in which certain Ab allotypic phenotypes or haplotypes predominate. In an example of this method, a subject is assigned to a defined population based on phenotype or genotype. The defined population may be one based on race or ancestral background, e.g., White, Black, or Asian. Once a subject is assigned to a defined population, an Ab is selected for administration to the subject from a set of Ab including at least a first Ab having the first allotypic phenotype or haplotype more common in the defined population and a second Ab of a second allotypic phenotype or haplotype less common in the defined population. The first Ab is then selected for administration to the subject on the basis that the subject will be more likely to express the first allotypic phenotype or haplotype than the second allotypic phenotype or haplotype. For example, in the Black population, the use of Abs of the G1m3 allotype is rare, while the use of G1m1, G1m17 is common. Thus use of an Ab of the G1m3 allotype would be less preferred than one of the G1m1, G1m17 allotype.

### Methods of Treating Subjects Who Have Developed an Anti-Antibody Response

In another aspect, the invention relates to a method of treating a subject who has developed an anti-Ab response against a previously administered Ab (e.g., a HAHA or HAMA response). In this method, administration of the previous Ab is discontinued and the subject is instead administered a second Ab having the identical variable region to the previously administered Ab but a different allotypic phenotype (preferably one expressed by the subject) than the previously administered Ab. Thus, any anti-allotypic reaction within the anti-antibody response is eliminated or reduced. This method is therefore particularly useful when an anti-antibody response is characterized by the presence of Abs in the subject that specifically bind to allotypic determinants of the previously administered Ab.

### Method of Selecting a Monoclonal Antibody Less Likely to Induce an Anti-antibody Response

Also within the invention is a method for selecting an Ab less likely to induce an anti-Ab response in a subject. The method can include first determining one or more (e.g., 1 2, 3, 4, 5, 6, 7, 8, 9, 10 or more) Ab allotypic phenotypes of the subject. An Ab (a) having one or more allotypic phenotypes of the subject and/or (b) having none of the allotypic phenotypes not expressed by the subject is then selected from a panel of different Ab having different allotypic phenotypes (and optionally with the same variable regions). The selected Ab is then administered to the subject.

### Pharmaceutical Compositions and Methods

The Ab compositions of the invention may be administered to animals or humans in pharmaceutically acceptable carriers (e.g., sterile saline), that are selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject.

For example, the Abs compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and or 0.2 micron filter); and/or dissolved in a solution including a microbicide (e.g., a detergent, an organic solvent, and a mixture of a detergent and organic solvent).

The compositions of the invention may be administered to animals or humans by any suitable technique. Typically, such administration will be parenteral (e.g., intravenous, subcutaneous, intramuscular, intrasternal, or intraperitoneal introduction). The compositions may also be administered directly to the target site by, for example, surgical delivery to an internal or external target site, or by catheter to a site accessible by a blood vessel. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. As is well known in the medical arts, dosage for any one animal or human depends on many factors, including the subject's size, body surface area, age, the particular composition to be administered, sex, time and route of administration, general health, and other drugs being administered concurrently. It is expected that an appropriate dosage for intravenous administration of antibodies would be in the range of about 0.01 to 100 mg/kg body weight.

### Kits

The invention also features kits for determining a subject's Ab allotypic phenotype such that the subject can be administered an Ab that matches or closely resembles an Ab allotypic phenotype expressed by the subject. An exemplary kit might include a plurality of probes that specifically identify Ab allotypic phenotypes in a biological sample isolated from a subject, other reagents such as buffers, containers such as test tubes or microtiter plates, positive and negative controls (e.g., Abs expressing a particular allotypic phenotype being screened for and Abs not expressing an allotypic phenotype not being screened for; or nucleic acids encoding Abs of an allotypic phenotype being screened for and nucleic acids encoding Abs not including an allotypic phenotype being screened for), and printed instructions for use. The probes might be polymerase chain reaction primers for specifically amplifying nucleic acids encoding an amino acid sequence corresponding to an allotypic phenotype being screened for or Abs that specifically bind an amino acid sequence corresponding to an Ab allotypic phenotype being screened for as described in more detail above.

An Ab-based probe can be used to detect the presence of an Ab of a particular allotypic phenotype using immunoassays such as ELISA, RIA, precipitin analysis, or Ouchterlony double diffusion analysis. Preferably such immunoassays are arranged to detect several (3, 4, 5, 6, or more) different Ab allotypic phenotypes in a single assay. For example, different wells of a microtiter plate can be coated with different (e.g., 2, 3, 4, 5, 6, 7, or more) capture Abs specific for different Ab allotypic phenotypes such that a single biological sample can be simultaneously screened for different allotypic phenotypes in a single ELISA or RIA. Similarly, in an Ouchterlony assay, the biological sample can be added to the center well and several different Abs each specific for a different allotypic phenotype can be added to individually to different wells that encircle the central well. Alternatively, a hemagglutination inhibition assay as described above might be included in a kit to detect the presence of an Ab of a particular allotypic phenotype.

A kit for determining a subject's Ab allotypic phenotype might also include reagents for determining whether a biological sample from a subject contains nucleic acids encoding one or more (e.g., 1, 2, 3, 4, 5, 6 or more) Ab allotypic phenotypes. For example, the kit might include detectably labeled nucleic acids complementary to those nucleic acids that encode Abs of particular allotypic phenotypes, PCR primer sets that selectively amplify those nucleic acid regions that encode the amino acid sequences in an Ab specific to a particular allotypic phenotype, or restriction endonucleases which cleave nucleic acid sequences on an allotypic phenotype-specific basis (for RFLP analysis).

### Methods for Screening a Subject for the Presence of AAAbs

Use of Abs matched or closely resembling a subject's Ab allotypic phenotype is particularly important in those subjects that already have AAAbs. Thus, in one aspect, the invention relates to methods for screening a subject for the presence of AAAbs. In these methods, a biological sample can be obtained from a subject and then screened for the presence of AAAbs. Biological samples can include any that might contain AAAbs, e.g., blood, plasma, serum, lymph, saliva, urine, cerebrospinal fluid, and synovial fluid. Any suitable method for obtaining a biological sample might be employed. For example, a peripheral blood sample can be obtained by venipuncture. Whole blood might be separated into a cell fraction (e.g., buffy coat), plasma, or serum according to known techniques. In some cases, biological samples obtained from a subject can be further processed to purify Ab-containing portions or cells having a nucleic acid sequences encoding Abs.

The presence of AAAbs in a biological sample can be determined by any suitable method. For example, an Ab of the allotype to which an AAAb is specific can be used as a probe which is contacted to the biological sample. Binding of the Ab probe to the AAAb can be detected as an indication that the subject has a specific AAAb. Such probe Abs can be used to detect a specific AAAb in a biological sample using a variety of immunoassays such as aggregate formation, precipitin analysis, Ouchterlony double diffusion analysis, ELISA (e.g., using the probe Ab as a capture Ab), and RIA (e.g., using the probe Ab as a capture Ab).

In a typical method, a biological sample such as blood, serum, or plasma is isolated from a subject. The isolated sample may be used directly or further processed to enrich Abs in the sample, e.g., by salt cuts, size exclusion chromatography, ion exchange chromatography, immunoaffinity chromatography (e.g., chromatography beads coupled to anti-human Ig antibodies can be used to isolate human Igs), and/or Protein A or Protein G chromatography. A capture Ab of the allotype which the AAAb being screened for would bind can be immobilized in wells of a microtiter plate. The biological sample potentially containing the AAAb is added to the wells and the wells are washed. A labeled Ab specific for the Abs of the subject (but not of the capture Ab) is then added to the wells. The wells are then washed, and the presence of the label in the wells is assessed. Presence of the label in a well indicates that the biological sample added to that well contained the AAAb. Alternatively, a hemagglutination assay might be used. For example, O+ red cells are coated with Igs of a single known allotype. If addition of a biological sample causes the coated cells to agglutinate, then that biological sample contains the screened for AAAb.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of selecting a human or humanized monoclonal antibody for administration to a human subject, the method comprising the steps of:
(a) determining the presence of a first antibody allotypic phenotype in the subject; and
(b) selecting a monoclonal antibody to be administered to the subject from a set of human or humanized monoclonal antibodies comprising at least a first monoclonal antibody comprising the first allotypic phenotype and a second monoclonal antibody comprising a second allotypic phenotype endogenously not present in the subject; and
(c) administering the first monoclonal antibody to the subject.

2. The method of claim 1, wherein the first monoclonal antibody does not comprise an allotypic phenotype not endogenously present in the subject.

3. The method of claim 1, wherein the first monoclonal antibody is of a first isotype and comprises a first variable region, and the second monoclonal antibody is of the first isotype and comprises the first variable region.

4. The method of claim 1, wherein the subject has rheumatoid arthritis.

5. The method of claim 1, wherein the subject has been previously transfused with heterologous blood.

6. The method of claim 1, wherein the subject is a female who has been pregnant.

7. The method of claim 1, wherein the subject is a child less than one year of age.

8. The method of claim 1, wherein the subject has been previously administered an antibody comprising the second allotypic phenotype.

9. A method of selecting a monoclonal antibody for administration to a human subject belonging to a defined population, the method comprising the steps of:
(a) determining a defined population that comprises the subject;
(b) selecting a monoclonal antibody for administration to the subject from a set of human or humanized monoclonal antibodies comprising at least a first monoclonal antibody comprising a first allotypic phenotype more common in the defined population and a second monoclonal antibody comprising a second allotypic phenotype less common in the defined population than the first allotypic phenotype; and
(c) administering the first monoclonal antibody to the subject.

10. The method of claim 9, wherein the first monoclonal antibody comprises a first haplotype more common in the defined population and the second monoclonal antibody comprises a second haplotype less common in the defined population than the first haplotype.

11. The method of claim 9, wherein the defined population is the White population.

12. The method of claim 9, wherein the defined population is the Black population.

13. The method of claim 9, wherein the defined population is the Asian population.

14. The method of claim 9, wherein the subject has rheumatoid arthritis; has been previously transfused with heterologous blood; is a female who has been pregnant; or is a child less than one year of age.

15. The method of claim 9, wherein the subject has been previously administered an antibody comprising the second allotypic phenotype.
